(19) United States
(12) Patent Application Publication
(10) Pub. No.: US 2005/0080384 A1
(43) Pub. Date: Apr. 14, 2005

(54) Illuminating syringe

(76) Inventor: Henry Edward Green JR., Pleasanton, CA (US)

Correspondence Address: Henry E. Green Jr., 5108 Corona Court, Pleasanton, CA, 94588, US

(21) Appl. No.: 10/960,909

(22) Filed: Oct. 12, 2004

Related U.S. Application Data
(60) Provisional application No. 60/510,108, filed on Oct. 10, 2003.

(51) Int. Cl. A61M 5/315

(52) U.S. Cl. 604/218

(57) ABSTRACT

A syringe having luminescent features to enhance readability. Said syringe having a central body portion that is a barrel having a proximal and distal end along a central longitudinal axis that acts as a reservoir for fluid inside the syringe. A plunger and coupled seal member can be engaged with the proximal end of the barrel creating a liquid tight reservoir inside the syringe. A plunger having a metal hook seated with a battery contacts a side of the battery. The metal hook is connected to a wire. A different wire contacts the opposite side of the battery. Both wires run along the longitudinal axis of the plunger to a light emitting diode. A tab is located between the battery and the metal hook in order to prohibit contact until ready to use. Illumination of barrel increases readability of demarcations on the barrel.

### CROSS-REFERENCE TO RELATED APPLICATIONS

This application claims the benefit of Provisional Patent Application Ser. No. 60/510,108 filed Oct. 10, 2003.

### FEDERALLY SPONSORED RESEARCH

Not Applicable

### SEQUENCE LISTING OR PROGRAM

Not Applicable

### BACKGROUND—FIELD OF INVENTION

This invention relates to medical instruments, specifically a syringe, which uses a light located adjacent to the seal to illuminate said demarcation on the syringe.

### BACKGROUND—DESCRIPTION OF PRIOR ART

Numerous patents discussing structure changes and demarcation coloration to help medical personnel have already been introduced to the market. Reviewing the U.S. Pat. No. 6,290,678, it concerns using different colors for the demarcations on the barrel of the syringe. However, this helps medical personnel see the demarcations in a well lit area; it does not aid medical personnel in a poorly lit conditions.

To overcome the shortcomings, the present invention provides an improved syringe to obviate, the aforementioned problem.

### BACKGROUND—OBJECTS AND ADVANTAGES

Several objects and advantages of the present invention are:

(a) to provide a syringe which is equipped with a light source which can be a variety of colors;

(b) to provide a syringe whose demarcations can be a variety of colors;

(c) to provide a syringe which will improve readability of demarcations;

(d) to provide a syringe which will reduce eyestrain of the handler of the syringe;

(e) to provide a syringe which will reduce reading errors due to medical delivery;

(f) to provide a syringe whose tab when removed will illuminate said demarcations; and

(g) to provide a syringe which will improve volume measurements in low lit areas.

Additional objects and advantages of the present invention are apparent from the drawings and specifications, which follow.

### SUMMARY

The Illuminating Syringe, refer to provisional patent application 60/510,108 filed on said date of Oct. 10, 2003.

The invention, an improved syringe, has a light emitting diode attached to the seal. This light emitting diode receives power from a circuit created by a battery, a metal hook, two wires connected from the battery to the light emitting diode all housed on a plunger.

Accordingly several objects and advantages of the invention are to provide an improved syringe, to provide means for making measurement of fluid inside of the barrel easier to perform, to enhance the readability of the demarcations.

### DRAWINGS

FIG. 1 is a perspective view of the illuminating syringe constructed in accordance with the invention.

FIG. 2 is a detailed perspective view of the illuminating syringe.

### REFERENCE NUMERALS

10 syringe

12 barrel

14 plunger

16 disc member

18 seal

20 flange

22 aperture

24 demarcations

26 needle

28 battery

30 metal hook

32 wire

34 light emitting diode

36 wire

38 tab

### DESCRIPTION

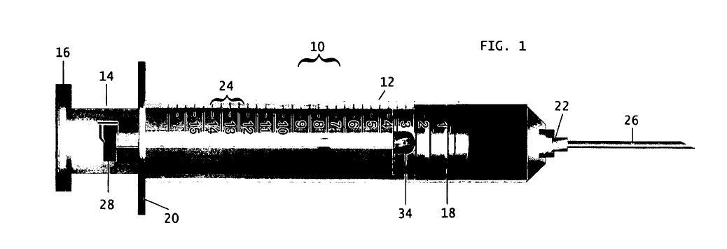

FIG. 1 shows an Illuminating Syringe 10 according to the present invention. The device consists of a central body portion that is a barrel 12 having proximal and distal ends along a central longitudinal axis that acts as a reservoir for fluid inside the syringe 10. The interior side wall of the barrel 12, or fluid reservoir, is preferably circular in shape, spaced evenly from the central axis and defines an inside diameter of the reservoir for holding fluid. The proximal end of the barrel portion 12 is open to receive a plunger 14 and coupled clear seal member 18. The distal end has an aperture 22 for connecting a cannula or needle 26 permitting fluid exchange with the interior reservoir of the barrel 12. The plunger 14 is a separate component from the syringe barrel 12 which consists of an elongated central body portion having a longitudinal axis and two perpendicular intersecting flat members; having a proximal end member and distal end member. Said proximal end member is a disc member 16. Said distal end member is a clear seal member 18 an light emitting diode 34.

When the plunger 14 is inserted in the proximal end of the barrel 12, a flange 20 located on the perimeter of the proximal end when operated in concert with the disc member 16 on the plunger 14 assist in the engagement of the plunger 14 and movement of same inside the syringe barrel 12.

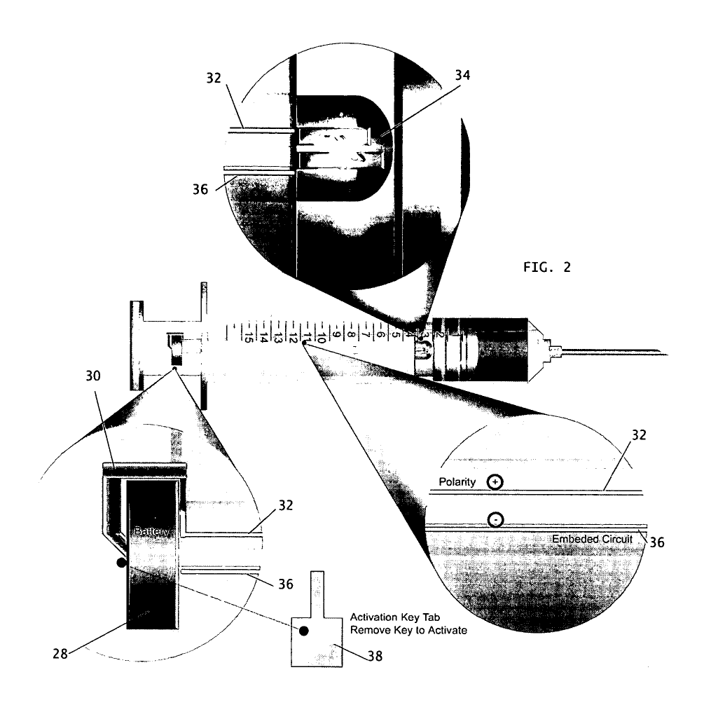

FIG. 2 is a detailed view of an Illuminating Syringe 10, showing the components of the plunger 14. Attached to said seal member 18 contains a fixed light emitting diode 34. The two perpendicular intersecting flat members at the proximal end of the plunger are cut to house a battery 28 and a metal hook 30. The metal hook 30 contacts one side of said battery 28, either positive or negative. Connected to the metal hook 30 is a wire 32, which runs along a side of the perpendicular intersecting flat members of the plunger 14. Said wire 32 is joined to the corresponding charged post of the light emitting diode 34. A wire 36 is connected to the other side of the battery 28. Said wire 36 runs along another side of the perpendicular intersecting flat members of the plunger 14. Said wire 36 is joined to the corresponding charged post of the light emitting diode 34.

When a tab 38 is located between metal hook 30 and battery 28 the circuit is blocked. The tab 38 can be manufactured from any thin type of material that prohibits an electrical current. Said tab 38 is large enough to prevent a connection between the metal hook 30 and battery 28. Said tab 38 must be long enough to protrude from the side of the plunger 14.

The side wall of the barrel includes demarcations 24 to measure the volume of the liquid contained within the syringe barrel 12. These demarcations 24 are used to determine the volume of the fluid inside the syringe barrel 12 as is required for delivery of the proper volume of medication or the like through the syringe 10. In this embodiment, the volume demarcations 24 extend around at least one-half of the body of the barrel 12.

It is the object of the demarcations 24 to provide a means for determining the volume of the fluid inside the syringe 10 in an easy and accurate manner even when the user may be somewhat visibly impaired or environmental conditions prohibit visibility. When the tab 38 is removed the demarcations 24 on the syringe 10 are easy to see and aid in the delivery of proper dosages of injected medications when used by a variety of individuals including diabetics with impaired eyesight, senior citizens and medical staff.

In the preferred embodiment volume demarcations 24 are applied to the side wall using an ink print printing process. To one skilled in the art, it is obvious there are numerous ways to print volume demarcations 24 on the syringe barrel 12 including printing with ink, embossed, applications or molding the volume demarcations 24 in the barrel 12 which may be later printed or altered by color application to effect volume demarcations 24 on the syringe 10.

One skilled in the art would also be aware that a myriad of ink or light emitting diode combinations could be used for the present invention. Although the black ink and blue light emitting diode is preferred for the volume demarcations 24, other ink and light emitting diode colors could be used; however, the ink and light emitting diode colors must be distinct enough to create the desirable effect of a contrast of the demarcations 24 on the syringe barrel surface 12 to aid in the reading of accurate measurements of the fluid contained therein. It should be noted that the present invention uses existing standard volume markings but improves the readability of the volume demarcations 24 by enhancing the same with the use of black ink and blue light emitting diode 34.

The syringe assembly 10 may be filled with liquid medication facilitated by the demarcations 24 of the present invention.

Operation

In operation one uses the syringe 10 in a normal manner with injecting or extracting fluids into the barrel 12 by pushing or pulling the disc member 16. The user, can, when desired, illuminate the syringe 10 by removing the tab 38 located beneath the disc member 16 on the plunger 14 (FIG. 2). When the tab 38 is removed, four effects increase the visibility of the syringe:

(1) The light emitting diode 38 receives power and illuminates the barrel 12 of the syringe 10.

(2) This increases the visibility of the demarcations on the barrel 12 for an easier reading.

(3) The visibility of the demarcations in any environment is improved.

(4) The possibility of error in injecting of extracting the incorrect amount of fluids is reduced.

Conclusion, Ramifications, and Scope

The syringe barrel 12 of the present invention may be constructed of a wide variety of rigid materials including thermoplastic materials or glass. A variety of commercially available inks and light emitting diodes may be used to create the illuminating contrast of the markings on the syringe barrel 12 and the seal member 18 on the plunger 14. Manufacturers of the syringe barrel 12 should employ only processes and products that permit the syringe 10 to be sterilized prior to use. The inks and light emitting diodes used on the present invention should also be amenable for sterilized as required for medical application. Therefore, the present invention provides a unique, useful, and reliable means for increasing the readability of the volume demarcations 24 on a syringe 10. Using ink and light emitting diode to create hyper indications of the volume demarcations 24 will aid in the accuracy of liquid medication dosage contained therein.

1. In a syringe, comprising:
A central body portion that is a barrel having a longitudinal axis and a side wall spaced from said axis defining an inside diameter and a reservoir for retaining fluid, said barrel having an open proximal end and a distal end having a passageway therethrough in fluid communication with said reservoir; the proximal end of the barrel is open to receive a plunger and coupled clear seal member; said plunger consists of an elongated central body portion having longitudinal axis, the proximal end of said plunger is cut to house a metal hook and a battery, a flat tab is situated between said metal hook and battery to prohibit contact, said metal hook having contact to a wire along the longitudinal axis of plunger, another wire having contact with said battery on its opposite side extends along the longitudinal axis of said plunger, said wires having contact to light emitting diode attached to said seal member, said side wall including a transparent portion having demarcations, whereby:
(a) during any environmental conditions, a user of the syringe can pull said tab activating the light emitting diode to receive power to illuminate the syringe in order to better read the demarcations.

2. The syringe plunger of claim 1 wherein said light emitting diode is a white, blue, Yellow, green and any contrasting color.

3. The syringe plunger of claim 1 wherein said seal has a light emitting diode means for reducing errors in volumetric readings.

4. The syringe barrel of claim 1 wherein said demarcations extend at least around one-half the circumference of said barrel.

5. The syringe barrel of claim 1 wherein said demarcations are at least one color that will contrast said light emitting diode.
